(19) United States
(12) Patent Application Publication
(10) Pub. No.: US 2009/0287003 A1
(43) Pub. Date: Nov. 19, 2009

(54) Process for the production of intermediates for making prostaglandin derivatives such as latanaprost, travaprost, and bimatoprost

(76) Inventor: Jiang Xing Chen, Shanghai (CN)

Correspondence Address: JUNEAU PARTNERS, P.O. BOX 2516, ALEXANDRIA, VA, 22301, US

(21) Appl. No.: 12/088,584

(22) PCT Filed: Sep. 29, 2006
(86) PCT No.: PCT/US06/38015

§ 371 Date: Oct. 15, 2008

Related U.S. Application Data
(60) Provisional application No. 60/721,575, filed on Sep. 29, 2005.
(60) Provisional application No. 60/752,600, filed on Dec. 22, 2005.

(51) Int. Cl. C07D 307/93; C07C 59/54

(52) U.S. Cl. 549/465; 562/471

(57) ABSTRACT

The subject matter of the invention is directed to a chemical process, namely, a process for the production of an intermediate compound used to make the pharmaceutical compound such as latanoprost.

### CROSS REFERENCE TO RELATED APPLICATIONS

This application is a related application of U.S. Provisional 60/721,575, filed 29Sep. 2005, herein incorporated by reference in its entirety.

### BACKGROUND

1. Field of the Invention

The subject matter of the invention is directed to a chemical process, namely, a process for the production of an intermediate compound used to make the pharmaceutical compound latanoprost.

2. Description of the Prior Art

Latanoprost is a topical medication used for controlling the progression of glaucoma or ocular hypertension, by reducing intraocular pressure. It works be increasing the outflow of aqueous fluid from the eyes. It is also known by the brand name of Xalatan Op™. The chemical formula for latanoprost is C₂₆H₄₀O₅. Latanoprost is a prostaglandin F2_analogue. Its chemical name is isopropyl—(Z)-7-[(1R,2R,3R,5S)3,5-dihydroxy-2-[(3R)-3-hydroxy-5-phenylpentyl]cyclopentyl]-5-heptenoate. Its molecular formula is C26H40O5 and its chemical structure is:

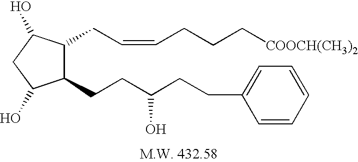

Latanoprost is a colorless to slightly yellow oil that is very soluble in acetonitrile and freely soluble in acetone, ethanol, ethyl acetate, isopropanol, methanol and octanol. It is practically insoluble in water. Latanoprost, an isopropyl ester prodrug, is hydrolyzed

by esterases in the cornea to the biologically active acid

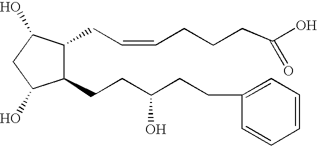

U.S. patents disclose the compound as well as its uses, specifically U.S. Pat. Nos. 4,599,353; 5,296,504 and 5,422,368. These patents also describe therein a process for the manufacture of latanoprost. Other patents describing related processes include: U.S. Pat. Nos. 6,353,014; 6,353,000; 6,235,779; and 4,021,425, all patents incorporated herein in their entirety. Many pending U.S. patent applications also describe process for production of prostaglandin derivatives (as they are known), including U.S. 20050209337 A1, and U.S. 20050038123 A1, all incorporated herein by reference in their entirety including especially conversion of the intermediate (3) to latanoprost or other derivatives.

Commercial intermediates are also available. Latanoprost lactol is available from Pharmatech International (Fairfield, N.J.) and can be converted to the free acid by Wittig reaction. Similarly, Latanoprost lactone diol (Pharmatech Int'l, Fairfield, N.J.) can be converted to the free acid of latanoprost by reduction with DIBAL followed by Wittig reaction with commercially available reagents.

However, processes for the production of these intermediates frequently involves the use of dangerous reagents and conditions, both of which add to the cost of production. In fact, beyond latanoprost, there are many valuable pharmaceutical compounds which have the same problems of safety and cost of producing their intermediates and final products.

Accordingly, there is a need for a more cost-effective and/or safer process for the production of latanoprost intermediates. There is also a need for a safer and/or more cost-effective process for making other compounds having a similar synthetic process as well.

### SUMMARY

Disclosed is a compound of this formula:

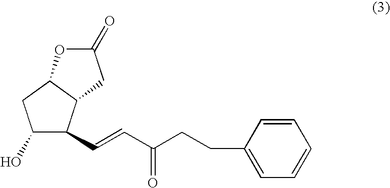

The present inventive subject matter provides a process for the production of an intermediate for making latanoprost, comprising the following steps along with appropriate reagents as set forth in the examples:

(i) converting Corey acid acetate to a Corey alcohol acetate

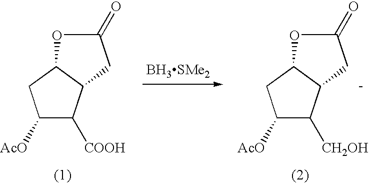

using a hydroboration reduction;
(ii) oxidizing the Corey alcohol acetate to a Corey aldehyde acetate,

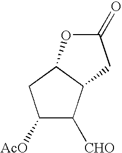

using pyridinium chlorochromate and dichloromethane;
(iii) converting the Corey aldehyde acetate into a Corey 1,1-dimethoxymethyl acetate,

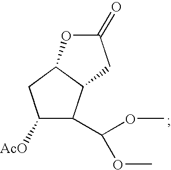

(iv) converting the Corey 1,1-dimethoxymethyl acetate into a Corey 1,1-dimethoxymethyl alcohol

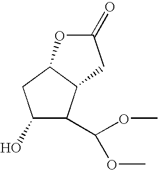

using anhydrous potassium carbonate;
(v) converting the Corey 1,1-dimethoxymethyl alcohol into a Corey aldehyde alcohol,

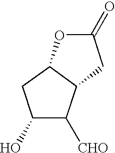

using anhydrous potassium carbonate, methanol, followed by 6N HCl, water, and acetone;
and,
(vi) converting the Corey aldehyde alcohol into compound 3, above, using an acid catalyzed Wittig reaction in a non-anhydrous environment.

Also disclosed is a method of producing prostaglandin derivatives using the process described herein as well as derivatives of the process as would be know to person of skill in the art and from reviewing the documents incorporated by reference herein in their entirety, especially processes which only require temperatures at about room temperature, i.e. 18-35° C.

Also disclosed is a specific method of producing latanoprost using the process described herein, especially processes which only require temperatures at about room temperature, i.e. 18-35° C.

### DETAILED DESCRIPTION OF THE PREFERRED EMBODIMENTS

### Definitions and Conventions

The definitions and explanations below are for the terms as used throughout this entire document including both the specification and the claims.

All temperatures are in degrees Celsius.

Bimatoprost refers to (Z)-7-[(1R,2R,3R,5S)-3,5-Dihydroxy-2-[1E,3S)-3-hydroxy-5-phenyl-1-pentenyl]cyclopentyl]-5-N-ethylheptenamide, and its molecular weight is 415.58. Its molecular formula is C₂₅H₃₇NO₄. Its chemical structure is:

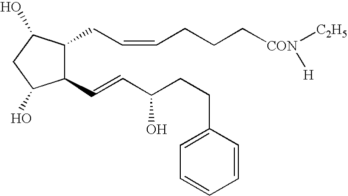

Brine refers to an aqueous saturated sodium chloride solution.

Chromatography (column and flash chromatography) refers to purification/separation of compounds expressed as (support, eluent). It is understood that the appropriate fractions are pooled and concentrated to give the desired compound(s).

HCl refers to hydrochloric acid. 6N HCl refers to the concentration of the acidity. The molecular weight of HCl 36.461 grams/mole. Since normality and molarity are the same for HCl (only one hydrogen), a 6N HCl solution is the same as a 6M HCl solution. Thus, 6×36.461 grams of HCl is 218.776 g/litre (w/w).

Latanoprost (XVI) refers to (5Z)-(9CI)-7-[(1R,2R,3R,5S)-3,5-dihydroxy-2-[(3R)-3-hydroxy-5-phenylpentyl]cyclopentyl]-5-heptenoic acid 1-methylethyl ester. It is also known as 17-phenyl-18,19,20-trinor-PF2aisopropyl ester.

PCC refers to pyridinium chlorochromate.

TLC refers to thin-layer chromatography.

THF refers to tetrahydrofuran.

Travaprost refers to a synthetic prostaglandin F_{2α} analogue. Its chemical name is isopropyl (Z)-7-[(1R,2R,3R,5S)-3,5-dihydroxy-2-[(1E,3R)-3-hydroxy-4-[(α,α,α-trifluoro-m-tolyl)oxy]-1-butenyl]cyclopentyl]-5-heptenoate. It has a molecular formula of C₂₆H₃₅F₃O₆ and a molecular weight of 500.56. The chemical structure of travoprost is:

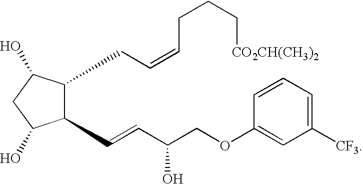

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

When solvent pairs are used, the ratios of solvents used are volume/volume (v/v).

When the solubility of a solid in a solvent is used the ratio of the solid to the solvent is weight/volume (wt/v).

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever.

Those skilled in the art will promptly recognize appropriate variations from the procedures both as to reactants and as to reaction conditions and techniques.

### Example

### Compound 3Synthesis

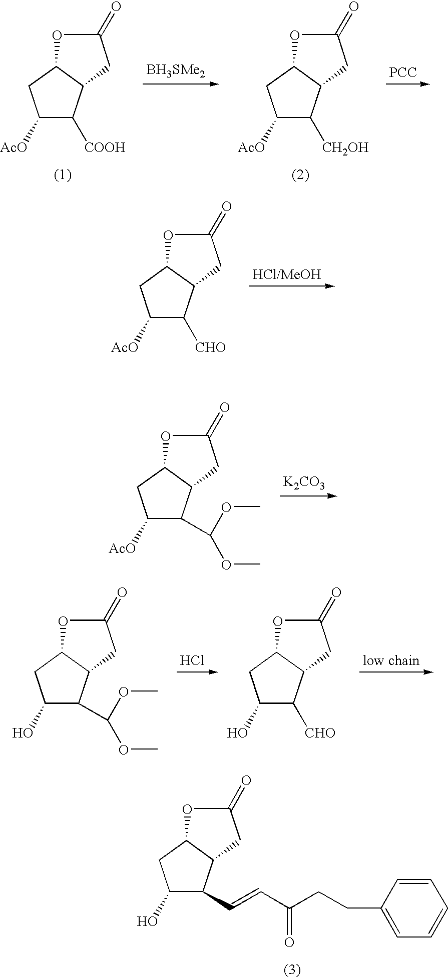

Corey Alcohol (2)

A solution containing Corey acid (1) (35 g, 0.15 mol) in dry tetrahydrofuran (THF) (200 ml) is cooled to 18° C., then the solution of dimethyl sulfide borane (BH₃—SMe₂) (2.0 mol/L in THF) (85 ml) is added drop by stir, the temperature of the reaction mixture was kept below 25° C. After added, the reaction mixture is stirred at room temperature for 2 hours (TLC monitoring). Then methanol (10 ml) was added and stir 10 min. Then evaporated in vacuo and dry with oil pump, the oily residue is used without purification in the next step.

Oxidation (3)

In a three neck bottom flask with mechanical stir, anhydrous sodium sulphate (100 g, 0.71 mol), dichloromethane (2000 ml), p-toluene sulfonic acid (PTS, 43 g, 0.23 mol) and PCC (45.5 g, 211.15 mmol) were added, and stir 0.5 hour, then a solution containing Corey alcohol which was prepared by last step reaction in dichloromethane (1000 ml) was added.

Subsequently, the reaction mixture is stirred vigorously at 30-35° C., for 2 hours (TLC monitoring). The precipitate was removed by filtration, washed with dichloromethane (2×100 ml), then a solution of HCl/methanol (105 ml) was added in the filtrate, the reaction mixture is stirred at room temperature for 10 hours, then anhydrous potassium carbonate (120 g) was added. The reaction mixture was stirred at room temperature for an additional 12 hours (TLC monitoring).

After filtering off the precipitate, the filtrate is evaporated under reduced pressure. The residue is dissolved in methanol (140 ml) and anhydrous potassium carbonate (28 g) was added, the reaction mixture was stirred at room temperature for 1 hour (TLC monitoring), then the solution is evaporated under reduced pressure and kept the bath temperature below to 30° C.

The residue is dissolved in water (30 ml), acetone (30 ml) and 6N HCl (120 ml), the reaction mixture was stirred at room temperature for 0.5 hour (TLC monitoring), then the low chain (8 g, 31.25 mmol) was added, the reaction mixture was stirred at room temperature for 2 hours (TLC monitoring), during this 2 hours, keep the PH=9-10 with potassium carbonate.

After reaction is finished, adjust the PH=7 by 6NHCl, and the reaction mixture was extracted with ethyl acetate (3×500 ml), The organic layer was washed with water (100 ml) and brine (3×100 ml),the water solution was extracted with ethyl acetate (3×100 ml), the organic layers were collected and dried on sodium sulphate (200 g) over night, then filtered, and evaporated in vacuo. The residue was purification by column chromatography on silica gel (500 g) and eluted by used an 7:3 mixture of ethyl acetate and hexane. After evaporating the solution, oil (5 g) were obtained. Total yield is 14.3% (w/w).

The second reaction is a named reaction—Wittig reaction, and in contrast to the present inventive subject matter, it is generally known (in all the paper, book and literature), that this reaction must be in dry solvent, mainly is dimethylsulfoxide (DMSO), and the reaction must be keep dry.

Compound 3 is contemplated to be useful for the production of final commercial products such as Latanoprost, as well as for making other useful precursors and intermediates useful for making Latanoprost such as hydrides and the like.

### Example

### Generic Process

Similar to the process above, a process for the production of an intermediate for prostaglandin derivatives, the process comprising: contacting a compound of formula (1)

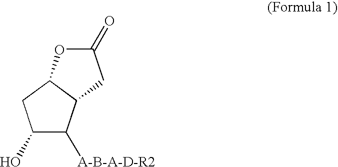

with Wittig reagents along with appropriate side chain, below, to produce a prostaglandin derivative,
wherein
B is a double bond
A is a carbon atom
D is a chain with 1-10, preferably 2-8, and especially 2-5, and particularly 3 carbon atoms, optionally interrupted by preferably not more than two hetero atoms (O, S or N), the substituent on each carbon atom being H, alkyl groups, preferably lower alkyl groups within 1-5 carbon atoms, a carbonyl group, or a hydroxyl group, whereby the substituent on C15 preferably being a carbonyl group; each chain D containing preferably not more than three hydroxyl groups or not more than three carbonyl group.
R2 is H, or a ring structure such as a phenyl group which is unsubstituted or has at least one substituent selected from C1-C5 alkyl groups, C1-C4 alkoxy groups, trifluoromethyl groups, C1-C3 aliphatic acylamino groups, nitro groups, halogen stoms, and phenyl group; or an aromatic heterocyclic group having 5-6 ring atoms, like thiazol, imidazole, pyrrolidine, thiophene and oxazole; or a cycloalkane or a cycloalkene with 3-7 carbon atoms in the ring, optionally substituted with lower alkyl groups with 1-5 carbon atoms;

The side chain which is used in the Witting reaction is defined by the following formula

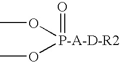

wherein
D is a chain with 1-10, preferably 2-8, and especially 2-5, and particularly 3 carbon atoms, optionally interrupted by preferably not more than two hetero atoms (O, S or N), the substituent on each carbon atom being H, alkyl groups, preferably lower alkyl groups within 1-5 carbon atoms, a carbonyl group, or a hydroxyl group; and,
R2 is H, or a ring structure such as a phenyl group which is unsubstituted or has at least one substituent selected from C1-C5 alkyl groups, C1-C4 alkoxy groups, trifluoromethyl groups, C1-C3 aliphatic acylamino groups, nitro groups, halogen stoms, and phenyl group; or an aromatic heterocyclic group having 5-6 ring atoms, like thiazol, imidazole, pyrrolidine, thiophene and oxazole; or a cycloalkane or a cycloalkene with 3-7 carbon atoms in the ring, optionally substituted with lower alkyl groups with 1-5 carbon atoms; and,
the Corey acid which is used in the Wittig reaction is defined by the following formula

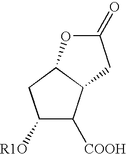

wherein
R1 is a acyl with 1-10, preferably 2-8, and especially 2-5, carbon atoms.

This genus produced by the generic process is contemplated to be useful for the production of final commercial products such as Travoprost and Bimatoprost.

It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

What is claimed is:

1. A compound of this formula:

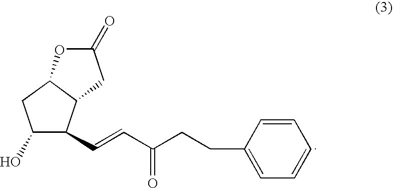

2. A process for the production of an intermediate for making latanoprost, comprising the following steps along with appropriate reagents as set forth in the examples:
(i) converting Corey acid acetate to a Corey alcohol acetate 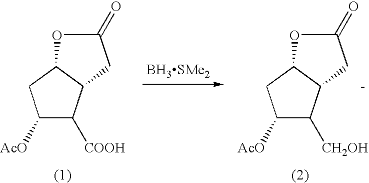
using a hydroboration reduction;
(ii) oxidizing the Corey alcohol acetate to a Corey aldehyde acetate, 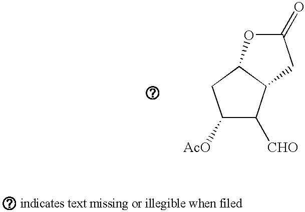
using pyridinium chlorochromate and dichloromethane;
(iii) converting the Corey aldehyde acetate into a Corey 1,1-dimethoxymethyl acetate, 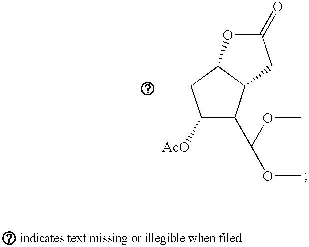
(iv) converting the Corey 1,1-dimethoxymethyl acetate into a Corey 1,1-dimethoxymethyl alcohol 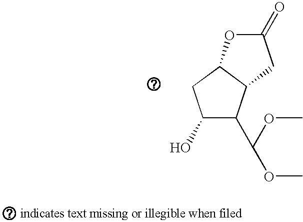
using anhydrous potassium carbonate;
(v) converting the Corey 1,1-dimethoxymethyl alcohol into a Corey aldehyde alcohol, 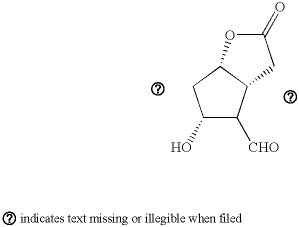
using anhydrous potassium carbonate, methanol, followed by 6N HCl, water, and acetone; and,
(vi) converting the Corey aldehyde alcohol into compound 3, above, using an acid catalyzed Wittig reaction in a non-anhydrous environment.

3. The process of claim 2, wherein the reaction temperatures of the process range from about 18° C. to about 35° C.

4. A method of producing prostaglandin derivatives, comprising:
contacting compound (3) 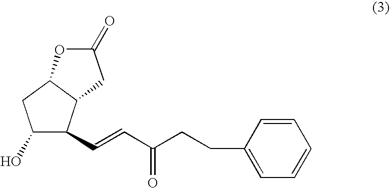
with Wittig reagents along with appropriate side chains to produce a prostaglandin derivative.

5. A method of producing latanoprost, comprising: contacting compound (3) 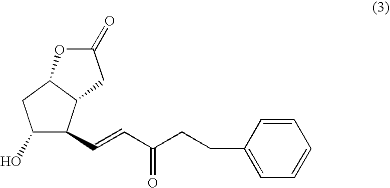
with reagents to produce latanoprost.

6. A process for the production of an intermediate for prostaglandin derivatives, comprising: (i) contacting a compound of formula (1) 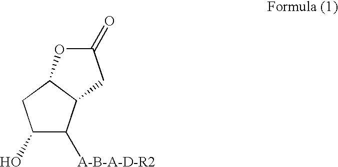
wherein
B is a double bond;
A is a carbon atom;
D is a chain with 1-10, preferably 2-8, and especially 2-5, and particularly 3 carbon atoms, optionally interrupted by preferably not more than two hetero atoms (O, S or N), the substituent on each carbon atom being H, alkyl groups, preferably lower alkyl groups within 1-5 carbon atoms, a carbonyl group, or a hydroxyl group, whereby the substituent on C15 preferably being a carbonyl group; each chain D containing preferably not more than three hydroxyl groups or not more than three carbonyl group; and
R2 is H, or a ring structure such as a phenyl group which is unsubstituted or has at least one substituent selected from C1-C5 alkyl groups, C1-C4 alkoxy groups, trifluoromethyl groups, C1-C3 aliphatic acylamino groups, nitro groups, halogen stoms, and phenyl group; or an aromatic heterocyclic group having 5-6 ring atoms, like thiazol, imidazole, pyrrolidine, thiophene and oxazole; or a cycloalkane or a cycloalkene with 3-7 carbon atoms in the ring, optionally substituted with lower alkyl groups with 1-5 carbon atoms;
with Wittig reagents along with a chemically appropriate side chain and a Corey Acid to produce a prostaglandin derivative;
wherein the chemically appropriate side chain used in the Witting reaction is defined by the following formula 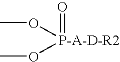
wherein
D is a chain with 1-10, preferably 2-8, and especially 2-5, and particularly 3 carbon atoms, optionally interrupted by preferably not more than two hetero atoms (O, S or N), the substituent on each carbon atom being H, alkyl groups, preferably lower alkyl groups within 1-5 carbon atoms, a carbonyl group, or a hydroxyl group;
R2 is H, or a ring structure such as a phenyl group which is unsubstituted or has at least one substituent selected from C1-C5 alkyl groups, C1-C4 alkoxy groups, trifluoromethyl groups, C1-C3 aliphatic acylamino groups, nitro groups, halogen stoms, and phenyl group; or an aromatic heterocyclic group having 5-6 ring atoms, like thiazol, imidazole, pyrrolidine, thiophene and oxazole; or a cycloalkane or a cycloalkene with 3-7 carbon atoms in the ring, optionally substituted with lower alkyl groups with 1-5 carbon atoms; and
wherein the Corey acid which is used in the Wittig reaction is defined by the following formula 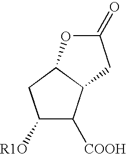
Wherein
R1 is a acyl with 1-10, preferably 2-8, and especially 2-5, carbon atoms.
